(19) United States
(12) Patent Application Publication
(10) Pub. No.: US 2023/0414887 A1
(43) Pub. Date: Dec. 28, 2023

(54) DISPLAY OF THE NO DOSE BY AN NO SUPPLY DEVICE IN PAUSE MODE

(71) Applicant: INOSYSTEMS, Antony (FR)

(72) Inventors: Yann BLANDIN, Bagneaux (FR); Mary SCHMITT, Bagneaux (FR); Nathan PROUVEZ, Antony (FR); Frederic MARCHAL, Antony (FR)

(21) Appl. No.: 18/213,711

(22) Filed: Jun. 23, 2023

(30) Foreign Application Priority Data
Jun. 24, 2022 (FR) 2206335

(51) Int. Cl. A61M 16/00; A61M 5/315

(52) U.S. Cl. CPC A61M 16/0069; A61M 16/0003; A61M 5/31553; A61M 2016/0027; A61M 2016/0039; A61M 2202/0208

(57) ABSTRACT

The invention relates to an NO supply device (1) comprising an internal passage for conveying a gaseous mixture NO/N₂; valve means; control means; dose selection means; memory storage means for storing the dose of NO to be administered; means (120) for starting treatment, and means for stopping treatment in order to pause or definitively discontinue a treatment by NO; and a graphic display (10) for displaying the NO dose before the commencement of the treatment or during the treatment by NO. The graphic display is further configured to continue to display the NO dose administered (110) to the patient, during a downtime (dt) in which the treatment by NO is interrupted temporarily.

### CROSS-REFERENCE TO RELATED APPLICATIONS

This patent application claims priority to French Application No. 2206335, filed Jun. 24, 2022, and the entire contents of which are incorporated herein by reference.

### BACKGROUND

The invention relates to a device or apparatus for supplying gaseous nitric oxide (NO), in particular a mixture of NO and nitrogen (N₂), and an installation for administering NO to a patient, which installation comprises such an NO supply device.

Inhaled nitric oxide, or NOi, is a gaseous medicament commonly used to treat patients suffering from acute pulmonary arterial hypertension, particularly pulmonary vasoconstrictions in adults or children, including the newborn (PPHN), as described for example in EP-A-560928 or EP-A-1516639.

An installation for implementation of a treatment by NOi, commonly known as an installation for administering NO, conventionally comprises one or more cylinders of NO/N₂ mixture which feed an NO supply device, which delivers the NO/N₂ mixture at a controlled flowrate, a respiratory assistance apparatus, also known as a medical ventilator, for supplying a respiratory gas containing at least 21% by volume of oxygen, such as an O₂/N₂ mixture or air, to which is added the NO (i.e. the NO/N₂ mixture) supplied by the NO supply device, circuit elements, for example one or more flexible ducts, to convey the gaseous flows between these different items of equipment and to the patient, and a respiratory interface, such as an endotracheal tube, to supply the gaseous mixture containing the NO to the patient. It is also possible to provide a gas humidifier in order to humidify the gaseous mixture before it is administered to the patient. An installation of this type is illustrated schematically in FIG. 1.

Normally, the NO/N₂ mixture delivered by the NO supply device is injected into the respiratory flow containing at least 21% by volume of oxygen (i.e. air or O₂/N₂ mixture) which is obtained from the medical ventilator before being administered by inhalation to the patient in the form of a final respiratory mixture (i.e. NO/N_{2/02} or NO/N₂/air mixture) generally containing a few tens of ppmv of NO (ppm in volume) and at least 21% by volume of oxygen O₂, for example of the order of 1 to 80 ppmv of NO, the remainder being substantially nitrogen (N₂).

An installation of this type for administering NO is used in a hospital environment in order to administer the treatment by NOi and thus care for patients who need to inhale NO in order to treat their pulmonary arterial hypertension. Examples of such NO administration installations are given in documents WO-A-2012/094008, US-A-2015/320951, US-A-2015/273175, JP-A-H11192303, WO-A-02/40914 and US-A-2003/116159.

In particular, NO-based medicaments are indicated in the treatment of patients suffering an acute episode of pulmonary arterial hypertension, which is a serious clinical situation and is life-threatening for the patients.

In the course of treatment by NOi, the NO has to be supplied at a precise dosage and preferably without interruption between the start and the end of the treatment by NOi.

However, during the course of treatment by NOi, it may be necessary or indeed essential to pause the administration system for a time, for example when the patient is temporarily disconnected from the ventilation system in order to undergo other medical treatments, such as bronchial aspiration or the like.

Nowadays, the known devices for administering inhaled NO display the NO dose in the process of being administered, that is to say the desired NO dose that has been chosen by the medical personnel and that is delivered during the actual treatment.

However, a pause in the operation of these known NO administration devices requires that the NO dose to be administered to the patient is set to zero, otherwise NO continues to be supplied by the NO supply device throughout the duration of the pause, typically for several minutes, or tens of minutes, which of course would not be desirable.

Thereafter, when the treatment is resumed, the medical personnel once again have to indicate to the NO administration device the desired dose of NO that is to be administered during the remainder of the treatment. However, this way of proceeding is unsatisfactory, since it poses risks to the patients. In fact, when setting or even prescribing a dose, the medical team may make a mistake if they have forgotten the dose that was set before the NO administration device was paused.

When the NO administration is resumed, that is to say at the end of the pause, the patients are exposed to the risk of receiving an NO dose that is not in accordance with the prescription.

A problem therefore is to be able to avoid or limit errors in the setting or prescribing of the NO dose by the medical personnel at the time of resumption of a treatment by NO that has been paused typically for several minutes, or tens of minutes, so as to reduce the risks of the patient being administered an NO dose that is not in accordance with his or her prescription.

### SUMMARY

A solution according to the invention therefore concerns an NO supply device comprising:
an internal passage for conveying a gaseous flow containing NO, typically a gaseous mixture NO/N₂,
valve means for controlling the gaseous flow in the internal passage,
control means for controlling at least the valve means,
dose selection means configured to allow a user to choose or adjust an NO dose to be administered,
memory storage means configured to store at least said NO dose to be administered,
means for starting treatment, which means are actuatable by the user and cooperate with the control means in order to initiate or resume a treatment by NO,
means for stopping treatment, which means are actuatable by the user and cooperate with the control means in order to pause or definitively discontinue a treatment by NO, and
a graphic display configured to display, before the commencement of the treatment by NO, the NO dose to be administered, and to display, during the treatment by NO, the dose administered to the patient.

According to the invention, the graphic display is further configured to continue to display the NO dose having been administered to the patient, during a downtime (dt) in which the treatment by NO is interrupted temporarily in response to an actuation, by the user, of the means for stopping treatment.

Depending on the embodiment considered, the device according to the invention may comprise one or more of the following features:
the control means are furtherconfigured to control the valve means in order to supply a gaseous flowrate corresponding to the NO dose to be administered during the treatment by NO, in response to the actuation, by the user, of the means for starting treatment.
the control means are further configured to control the valve means in order to temporarily interrupt all gaseous flow during the downtime (dt), in response to the actuation, by the user, of the means for stopping treatment.
the control means are configured to control the the graphic display to display the NO dose to be administered, before and during the treatment by NO and during the downtime (dt) in which the treatment by NO is interrupted temporarily.
the control means are configured to control the graphic display to continue to display, during the downtime (dt), the NO dose having been administered to the patient just before (i.e. immediately before) actuation, by the user, of the means for stopping treatment, that is to say before a pause.
the control means are configured to control the graphic display to display an adjusted NO dose to be administered to the patient, after the downtime (dt), said adjusted NO dose being chosen or adjusted by the user, during the downtime (dt), via the dose selection means.
the downtime (dt) has a duration of less than or equal to 30 minutes, preferably between about 1 and 20 minutes.
the memory storage means are configured to store the last NO dose administered to the patient immediately before the commencement of the downtime (dt), that is to say before a pause is made.
the means for starting treatment and the means for stopping treatment comprise one and the same actuation element, which is actuatable by the user.
alternatively, the means for starting treatment and the means for stopping treatment comprise separate actuation elements, which are actuatable by the user.
the one or more actuation elements comprise one or more keys, buttons, rotary selectors, cursors or the like.
the one or more actuation elements comprise one or more virtual selection keys displayed on the graphic display.
the graphic display is configured to display one or more virtual keys.
the graphic display is a touch-controlled digital display screen.
the control means comprise at least one (micro)processor.
the dose selection means comprise at least one touch-sensitive selection key displayed on the display screen.
the display screen is configured to display said at least one touch-sensitive selection key, preferably several touch-sensitive selection keys.
the one or more touch-sensitive selection keys are configured to allow a choice to be made between a number of proposed NO contents or, alternatively, they comprises “+” and “−” keys making it possible to increase or decrease a given NO value in stages, for example 0.1 ppmv or 1 ppmv, or similar (e.g. 2 by 2 ppmv, 3 by 3 ppmv, or 5 by 5 ppmv, etc.).
said desired NO content, which is chosen or selected by the user by acting on said touch-sensitive selection key, is supplied to the control means, preferably by digital action.
the selection or setting of the desired NO dose is carried out by the user's finger pressing on the one or more touch-activated selection keys displayed on the display screen.
the NO dose selection means are configured to allow the user to set or select a desired NO content of between 0.1 et 80 ppmv.
the display screen provides a display in colour or in black and white.
the control means are configured to control the one or more displays on the graphic display, i.e. the information display screen.
the control means comprise one or more (micro)processors, for example a microcontroller.
the control means comprise at least one electronic board comprising said at least one microprocessor.
the control means comprise at least one (micro)processor implementing at least one algorithm, for example for data processing, calculation or similar.
the one or more virtual keys displayed on the graphic display are touch-sensitive keys.
the graphic display is a touch panel.

According to another aspect, the invention also relates to an installation for administering therapeutic gas containing NO to a patient (P), comprising an NO supply device according to the invention, in particular as described above, supplied with NO/N₂ mixture by at least one pressurized gas container and with oxygen by a pressurized oxygen container, said NO supply device supplying an NO/N₂ mixture to a respiratory gas circuit connected to a medical ventilator supplying an O₂/N₂ mixture or air.

Depending on the embodiment considered, the therapeutic gas administration installation according to the invention can comprise one or more of the following characteristics:
the NO supply device can comprise a main outlet supplying the NO/N₂ mixture to the respiratory gas circuit connected to the medical ventilator, preferably via an NO injection duct or NO injection line, that is to say a flexible hose or similar
said at least one pressurized gas container contains the NO/N₂ mixture.
said at least one pressurized gas container contains an NO/N₂ mixture containing from 100 to 1000 ppmv of NO, the remainder being nitrogen.
the pressurized oxygen container contains medical oxygen.
the one or more containers are gas cylinders.
the one or more gas containers contain an NO/N₂ mixture or medical oxygen at a pressure of at least 150 bar, or at least 180 bar.
the respiratory gas circuit comprises (at least) an inhalation branch and an exhalation branch.
the inhalation branch and the exhalation branch are connected fluidically to each other via a joining piece, such as a Y-piece.
the joining piece is connected fluidically to a respiratory interface, such as an endotracheal tube, a breathing mask or spectacles for oxygenation.
the inhalation branch and the exhalation branch comprise flexible hoses.
a gas humidifier is arranged on the respiratory gas circuit, in particular on the inhalation branch.
the inhalation branch comprises a flowrate sensor which is connected electrically and/or via tubing to the NO supply device, in particular to the control means.
the flowrate sensor is arranged on the inhalation branch upstream from the NO injection site.
the flowrate sensor is a mass-flow sensor or a differential pressure sensor.
a gas sampling line fluidically connects the NO supply device to the respiratory gas circuit, preferably in proximity to the joining piece, i.e. the Y-piece.
the medical ventilator and the NO supply device are powered electrically by at least one source of electric current.
the NO supply device can comprise a backup outlet, i.e. a secondary outlet, for example a port or orifice, which may be connected fluidically to a manual insufflation bag.

Generally speaking, in the frame of the invention, the terms “means” are considered equivalent and substitutable for the terms “device” or the like. For example, the terms “valve means” are considered equivalent and substitutable for the terms “valve device”, the terms “pilot means” are considered equivalent and substitutable for the terms “pilot device”, etc.

The invention also concerns a method for therapeutically treating a patient, wherein a device for supplying NO according to the invention and/or an installation for administering therapeutic gas containing NO to a patient (P) comprising an NO supply device according to the invention, in particular as described above, is used for administering by inhalation a therapeutic gas containing nitric oxide or NO (i.e. a gaseous drug) to the patient to be treated, said patient suffering from a pulmonary arterial hypertension, especially pulmonary vasoconstrictions.

Depending on the embodiment, the method of the invention may comprise one or more of the following features:
the patient is an adult, adolescent, child, neonate or infant.
the patient has acute pulmonary hypertension.
the patient has PPHN, i.e. Persistent Pulmonary Hypertension of the Newborn.
the pulmonary hypertension is perioperative and associated with cardiac surgery.
therapeutic gas containing nitric oxide (NO) is administered at an amount of less than 40 ppmV.
the therapeutic gas contains nitric oxide (NO), nitrogen and oxygen (at least approximately 21% vol.), and possibly inevitable impurities.
the therapeutic gas containing nitric oxide (NO) is administered via a tracheal intubation tube or the like.
the NO supply device is fed by at least one pressurized gas container, e.g. a gas cylinder, containing an NO/N₂ mixture containing from 100 to 1000 ppmv of NO, the remainder being nitrogen.
the gas administration installation comprises a medical ventilator supplying a gas containing oxygen (at least approximately 21% vol.), for example an O₂/N₂ mixture or air.

### BRIEF DESCRIPTION OF THE DRAWINGS

The invention will now be better understood from the following detailed description given as a non-limiting example and with reference to the appended figures, in which:

FIG. 1 shows an embodiment of an installation for administering therapeutic gas to a patient, incorporating an NO supply device according to the invention,

FIG. 2 shows an embodiment of the graphic display of an NO supply device according to the invention, showing the information displayed during a treatment by NO, for example by means of the installation of FIG. 1, and

FIG. 3 shows an embodiment of the graphic display of an NO supply device according to the invention, showing the information displayed during a downtime in which the treatment by NO is interrupted temporarily.

### DETAILED DESCRIPTION

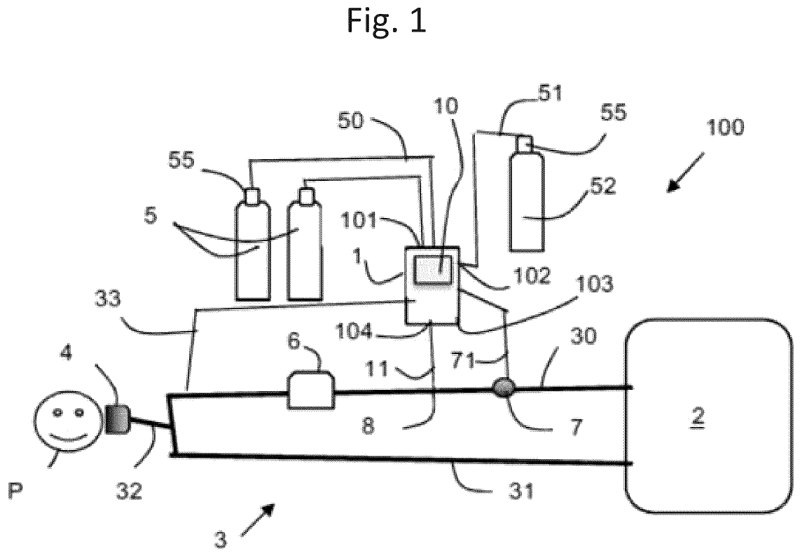

FIG. 1 shows schematically an embodiment of an installation 100 for administering therapeutic gas, i.e. a gaseous mixture based on NO, typically an NO/O₂/N₂ mixture, to a patient P, incorporating the NO supply device 1 according to the present invention.

More specifically, in this case it comprises two pressurized gas containers 5, arranged in parallel, each containing a gaseous mixture of NO and nitrogen (N₂), i.e. an NO/N₂ mixture, typically containing from 250 to 1000 ppmv of NO and nitrogen (N₂), which is also subjected to a pressure that can be as much as 180 bar or more, for example an NO/N₂ mixture, containing 450 ppmv or 800 ppmv of NO. Gas containers 5 of this type are commonly known as NO cylinders 5.

The NO cylinders 5 supply the NO/N₂ mixture to the NO supply device 1 according to the invention. They are connected fluidically to the gas supply device 1 by NO supply lines i.e. gas piping, such as flexible hoses or the like. Each NO supply line 50 is connected to an NO input port 101 of the NO supply device 1, in order to supply a main gas circuit 200 inside the housing 103 of the NO supply device 1.

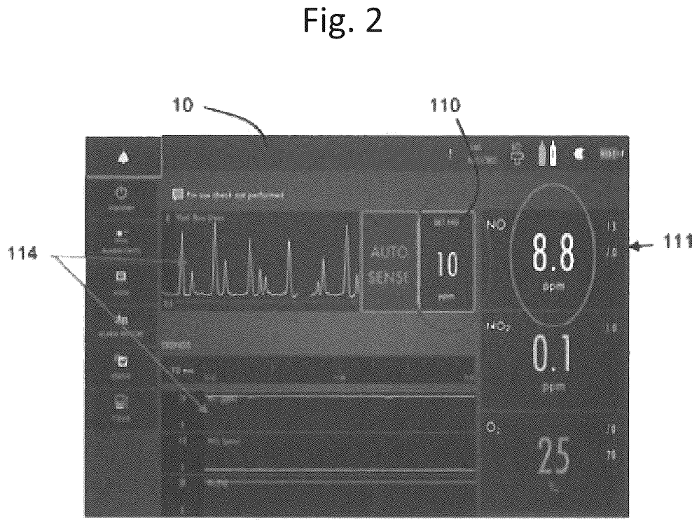

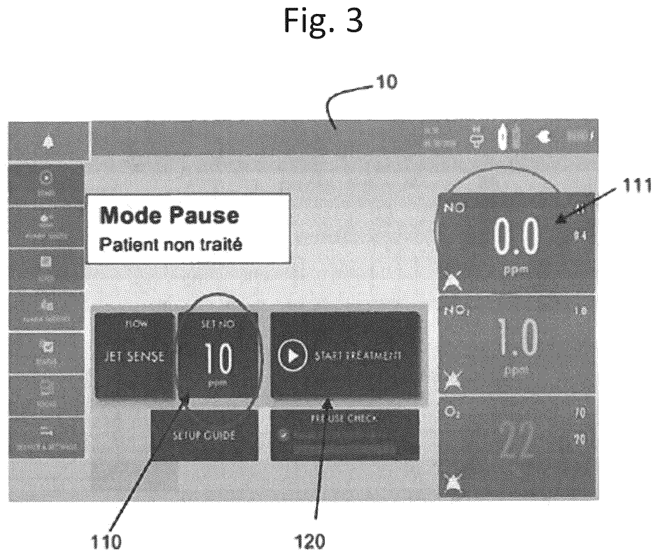

The housing 103 of the NO supply device 1 also comprises a graphic display 10, such as a touch screen, configured to display various items of information, notably the NO doses, as is explained below and illustrated in FIG. 2 and FIG. 3.

The NO supply device 1 also comprises an oxygen input port 102 which is connected fluidically, via an oxygen feed line 51, such as a flexible hose or the like, to a source of oxygen, for example a pressurized oxygen container 52, typically an O₂ cylinder, or, alternatively, the hospital network, i.e. oxygen supply piping arranged in the hospital building where the patient P is being cared for.

The NO cylinders 5 and the O₂ cylinder 52 are equipped with a gas distribution valve preferably incorporating gas expansion means, i.e. an RDI or valve with an integrated expansion device, so as to be able to control the flowrate and/or the pressure of the gas which they deliver. The gas distribution valve 55 is preferably protected against impacts by a protective cowl.

In addition, the installation 100 also comprises a medical ventilator 2, that is to say a respiratory assistance apparatus, which supplies a flow of respiratory gas containing at least 21% by volume of oxygen, such as air or an oxygen/nitrogen mixture (N₂/O₂), to the patient P.

The medical ventilator 2 is fluidically connected to the patient P via a respiratory gas circuit 3 which in this case has two respiratory branches 30, 31, since it comprises an inhalation branch 30, that is to say a gas supply line which serves to convey the respiratory gas to the patient P, and an exhalation branch 31 which serves to recuperate the gas enriched with CO₂ exhaled by the patient P.

The two respiratory branches 30, 31 are typically flexible hoses made of polymer or the like. The two respiratory branches 30, 31 are on the one hand connected to the medical ventilator 2 and on the other hand connected to each other at a joining piece 32, typically a Y-piece, which is in fluid communication with a respiratory interface 4 which supplies gas to the patient P, such as an endotracheal tube or the like.

It will be appreciated that the medical ventilator 2 and the NO supply device 1 are normally powered electrically by one or more sources of electric current, in particular their components which require electrical energy in order to operate, in particular the means for controlling the NO supply device 1 and the system for controlling the medical ventilator 2, i.e. an electronic board with a microprocessor/microprocessors, or any other component, in particular the motorized internal turbine which supplies the flow of air or the like, i.e. the respiratory gas. The source of electric current can be the mains (110/220V) and/or an electric battery, which is preferably rechargeable

As can be seen, the NO supply device 1 makes it possible to inject the NO/N₂ mixture into the inhalation branch 30, via an NO injection duct 11 which opens into the inhalation branch 30 at an injection site 8, such as to provide there a mixture of the flow of NO/N₂ and of the flow of respiratory gas containing at least 21% O₂, i.e. air or an oxygen/nitrogen mixture, delivered by the medical ventilator 2.

The NO supply device 1 comprises a main output orifice 104 situated at the outlet of its main gas circuit via which the flow of NO/N₂ exits from the housing 103 of the NO supply device 1 and penetrates into the NO injection duct 11. The NO injection duct 11 is connected fluidically to the main output orifice 104, for example via a connector or the like.

The therapeutic gaseous mixture obtained thus contains oxygen (>21% by volume), nitrogen, and a concentration of NO which is variable and adjustable, typically of between 1 and 80 ppmv, as a result of the dilution which takes place during the mixing of the gaseous flows. It will be appreciated that inevitable impurities can be found in the gas, but they are not desirable, in particular when the flow of gas obtained from the ventilator 2 is atmospheric air rather than a mixture of O₂/N₂.

Advantageously, a gas humidifier 6 is also provided, which in this case is arranged on the inhalation branch 30 downstream from the injection site 8 and serves to humidify the flow of therapeutic gas, i.e. an NO/N₂/O₂ mixture, by the addition of water vapour, before it is inhaled by the patient P, which makes it possible to avoid or limit drying of the airways of the patient P during his or her treatment by inhalation of the gas.

According to another embodiment, the gas humidifier 6 could also be arranged upstream from the injection site 8.

Depending on the case, the exhalation branch 31, serving to receive the exhaled gases rich in CO₂, can comprise one or more optional components, for example a CO₂ elimination device, i.e. a CO₂ trap, such as a hot container or the like, which makes it possible to eliminate the CO₂ present in the gases exhaled by the patient, a filter or the like.

On the inhalation branch 30, upstream from the injection site 8, a flowrate sensor 7 is also provided, for example a mass-flow sensor or a differential pressure sensor, connected to the NO supply device 1, in particular to the control means of said NO supply device 1, via a line 71 for measurement of the flowrate of respiratory gas, serving to measure the flowrate of gas obtained from the ventilator 2 within the inhalation branch 30.

Determining this flowrate of the ventilator makes it possible in particular to regulate the passage of the NO through the NO supply device 1, in particular to be able to select the flowrate of NO/N₂ mixture to be injected according to the desired NO content, the composition of the NO/N₂ mixture obtained from the cylinders, and the flowrate of gas (i.e. air or air/O₂) obtained from the ventilator 2.

In addition, a gas sampling line 33 can also be provided, connecting the NO supply device 1 fluidically to the respiratory gas circuit 3, preferably in proximity to the Y-piece 32, for example at approximately 10 to 20 cm upstream from the Y-piece 32, serving to collect gas samples and to verify, by means of a gas analyser or the like, that they conform with the desired gaseous mixture that is to be administered to the patient P.

More specifically, the NO supply device 1 comprises an internal main gas circuit by which the NO/N₂ mixture entering via the gas input port(s) 101 is conveyed as far as the NO injection duct 11. This main gas circuit comprises means for controlling the flowrate of NO/N₂, namely valves, calibrated orifices etc., controlled by the control means of the NO supply device 1, typically one or more microprocessors arranged on an electronic board, the operation of which is explained hereinbelow. All these components are arranged in the housing 103, that is to say a rigid external shell.

The control means of the NO supply device 1 also control the displays on the display 10, such as a touch screen, preferably in colours.

Moreover, the NO supply device 1 can also comprise a backup circuit designed to deliver an adjustable O₂ flowrate and a fixed NO flowrate, so as to be able to ensure a supply of NO even in the case of a fault or the like.

According to the present invention, the NO supply device 1 further comprises dose selection means, such as a virtual key displayed on the display 10, which means are configured to allow a user to choose or adjust an NO dose to be administered, and memory storage means which are configured to store the NO dose to be administered, or indeed other items of information.

Moreover, it also comprises means 120 for starting treatment, which are actuatable by the user, such as a virtual key displayed on the display 10 as seen in FIG. 3, which means 120 cooperate with the control means in order to commence or resume a treatment by NO, by controlling the valve means so as to supply the desired dose of NO.

By analogy, it also comprises means for stopping treatment, which are actuatable by the user, such as a virtual key displayed on the display 10, which means also cooperate with the control means in order to pause or definitively discontinue a treatment by NO, by controlling the valve means to interrupt the passage and supply of NO/N₂ mixture to the ventilation circuit connected to the ventilator 2.

The graphic display 10, for its part, is configured to display, before the commencement of the treatment by NO, the NO dose to be administered (110; 111), or to display, during the treatment by NO, the dose administered to the patient P. The displays on the graphic display are controlled by the control means of the NO supply device 1 according to the invention.

However, according to the invention, the graphic display 10 is further configured to continue to display the NO dose having been administered 110 to the patient, throughout a downtime (dt) in which the treatment by NO is interrupted temporarily, that is to say during a pause that can last several minutes or tens of minutes, typically less than 45 minutes, for example up to 20 to 30 minutes. The pause begins as soon as the user actuates the means for stopping treatment.

FIG. 2 shows an embodiment of the graphic display 10 of the NO supply device 1 according to the invention, showing the information displayed during a treatment by NO, for example by means of the installation of FIG. 1.

As will be seen, during treatment of a patient by administration of NO, the display 10 displays various items of information useful to the medical personnel, in particular:
the NO dose to be administered (at 110) to the patient, here 10 ppm by volume for example, which is chosen before the start of treatment;
the NO dose actually administered (at 111) to the patient during the treatment by NO, here 8 ppm by volume for example, that is to say the NO dose after the NO/N₂ mixture supplied by the device 1 is mixed with the gas coming from the ventilator 2, such as an N₂/O₂ mixture or air;
the quantity of NO₂ formed, here 0.1 ppm by volume, and present in the final mixture administered to the patient; and
the oxygen content, here 25% by volume, present in the final mixture administered to the patient.

The display 10 of the device 1 also displays other information, for example curves 114 from monitoring the NO, O₂ and NO₂ contents during the treatment by administration of NO to the patient P.

In other words, the NO dose set by the medical personnel is permanently visible (at 110), when the administration of NO is in progress, and the graphic display 10 also displays the dose actually delivered (at 111), which is measured by the gas analyser supplied via the gas sampling line 33.

FIG. 3 shows the information displayed on the graphic display 10 of the NO supply device 1 according to the invention during a downtime in which the treatment by NO is interrupted temporarily, that is to say after treatment has been paused.

In this case, the NO dose supplied is equal to 0 ppm (at 111), but according to the invention the graphic display 10 continues, despite the pause, to display the NO dose (at 110) that has been administered to the patient during the treatment phase, here 10 ppmv.

This display (at 110) is provided throughout the downtime (dt) in which the treatment by NO is interrupted temporarily, after the user has actuated means for stopping treatment, such as a touch-sensitive key shown on the graphic display 10, such as a touch-sensitive screen.

Means 120 for starting treatment, such as a touch-sensitive key displayed on the graphic display 10, make it possible to resume, that is to say continue, the treatment by NO when the means is actuated by the user, that is to say when the user presses on it with a finger.

In this case, the medical personnel do not risk making a mistake when the treatment is resumed, since the last known dose of NO that was supplied to the patient is permanently displayed throughout the downtime and until the treatment is resumed.

Optionally, the medical personnel may modify the dose before restarting the treatment.

1. NO supply device (1) comprising:
an internal passage for conveying a gaseous flow containing NO, typically a gaseous mixture NO/N₂,
valve means for controlling the gaseous flow in the internal passage,
control means for controlling at least the valve means,
dose selection means configured to allow a user to choose or adjust an NO dose to be administered,
memory storage means configured to store at least said NO dose to be administered,
means (120) for starting treatment, which means are actuatable by the user and cooperate with the control means in order to initiate or resume a treatment by NO,
means for stopping treatment, which means are actuatable by the user and cooperate with the control means in order to pause or definitively discontinue a treatment by NO, and
a graphic display (10) configured to display, before the commencement of the treatment by NO, the NO dose to be administered (110; 111), and to display, during the treatment by NO, the dose administered to the patient,
characterized in that the graphic display is further configured to continue to display the NO dose administered (110) to the patient, during a downtime (dt) in which the treatment by NO is interrupted temporarily, in response to an actuation, by the user, of the means for stopping treatment.

2. Device according to claim 1, characterized in that the control means are configured to control the graphic display to display the NO dose to be administered, before and during the treatment by NO and during the downtime (dt) in which the treatment by NO is interrupted temporarily.

3. Device according to claim 1, characterized in that the control means are further configured to control the valve means in order to supply a gaseous flowrate corresponding to the NO dose to be administered during the treatment by NO, in response to the actuation, by the user, of the means for starting treatment.

4. Device according to claim 1, characterized in that the control means are further configured to control the valve means in order to temporarily interrupt all gaseous flow during the downtime (dt), in response to the actuation, by the user, of the means for stopping treatment.

5. Device according to claim 1, characterized in that the means (120) for starting treatment and the means for stopping treatment comprise one and the same actuation element, which is actuatable by the user.

6. Device according to claim 1, characterized in that the means (120) for starting treatment and the means for stopping treatment comprise separate actuation elements, which are actuatable by the user.

7. Device according to claim 5, characterized in that the one or more actuation elements comprise one or more virtual keys displayed on the graphic display (10).

8. Device according to claim 1, characterized in that the control means are configured to control the graphic display (10) to continue to display, during the downtime (dt), the NO dose having been administered (110) to the patient just before actuation, by the user, of the means for stopping treatment, that is to say before a pause.

9. Device according to claim 8, characterized in that the memory storage means are configured to store the last dose of NO administered to the patient immediately before the commencement of the downtime (dt).

10. Device according to claim 1, characterized in that the downtime (dt) has a duration of less than or equal to 30 minutes.

11. Device according to claim 7, characterized in that the one or more virtual keys displayed on the graphic display (10) are touch-sensitive keys.

12. Device according to claim 1, characterized in that the graphic display (10) is a touch panel.

13. Installation (100) for administering therapeutic gas containing NO to a patient (P), comprising an NO supply device (1) according to claim 1, supplied with NO/N₂ mixture by at least one pressurized gas container (5) and with oxygen by a pressurized oxygen container (52), said NO supply device (1) supplying an NO/N₂ mixture to a respiratory gas circuit (3) connected to a medical ventilator (2) supplying an O₂/N₂ mixture or air.

14. Installation according to claim 13, characterized in that said at least one pressurized gas container (5) contains an NO/N₂ mixture containing from 100 to 1000 ppmv of NO, the remainder being nitrogen.

15. Installation according to claim 13, characterized in that the respiratory gas circuit (3) comprises a flowrate sensor.
